(19) United States
(12) Patent Application Publication
(10) Pub. No.: US 2013/0110115 A1
(43) Pub. Date: May 2, 2013

(54) MINIMALLY INVASIVE METHOD AND DEVICES FOR REPAIRING LOOSENED PROSTHETIC IMPLANTS

(71) Applicant: Doranne M. Lackman, Philadelphia, PA (US)

(72) Inventor: Doranne M. Lackman, Philadelphia, PA (US)

(73) Assignee: Medulaplasty LLC

(21) Appl. No.: 13/281,836

(22) Filed: Oct. 26, 2011

(51) Int. Cl. A61B 17/58

(52) U.S. Cl. 606/94

(57) ABSTRACT

Minimally invasive methods of using a composition including bone cement to secure a hip or other prosthetic implant that has loosened following the initial implant surgery are disclosed. The methods include delivery of a composition including one or more bone cements alone, or in combination with one or more additives and/or fillers to the site of a loosened implant-bone interface. Devices suitable for use in the method are also described.

### FIELD OF THE INVENTION

The present application is generally related to minimally invasive methods of treating loosened joint implants.

### BACKGROUND OF THE INVENTION

Joint replacement surgery involves removing an arthritic or dysfunctional joint and replacing it with an orthopedic prosthetic implant. Hip replacement is second only to knee replacement as the most common joint replacement procedure. However, other joints including shoulders, fingers, ankles, and elbows can also be replaced. Total hip replacement is used to treat joint failure caused by osteoarthritis, rheumatoid arthritis, avascular necrosis, traumatic arthritis, protrusio acetabuli, certain hip fractures, benign and malignant bone tumors, arthritis associated with Paget's disease, ankylosing spondylitis and juvenile rheumatoid arthritis. The most common type of hip arthritis is osteoarthritis. Often referred to as “wear-and-tear” arthritis, this condition results in the wearing away of the normal smooth cartilage until bare bone is exposed. Hip replacement is a common treatment for pain relief and improvement in hip function, but is usually considered only once other therapies, such as physical therapy and pain medications, have failed.

The typical hip replacement implant includes three components: the acetabular cup, the femoral component and the articular interface. The acetabular cup is the component which is placed into the acetabulum (hip socket). During hip replacement surgery, cartilage and bone are scraped out of the acetabulum and the acetabular cup is attached using friction or cement. The acetabular cup can be a single piece or a modular piece including a shell and liner. During hip replacement surgery, the head of the femur is removed and the interior of the bone is reshaped to accept the femoral component. The femoral component, which also can be singular or modular unit, includes both the “ball” of the “ball-and-socket”, as well as a fermoral stem which is inserted down the hollow center of the femur to support the joint. The acetabular cup is typically composed of plastic or metal, or a combination of the two, and the fermoral component is typically composed of metal, though other materials such as ceramic are also sometimes used.

Hip replacements eventually wear out. Some studies show that implants can last more than 20 years, but in some patients, the prosthetics fail immediately after surgery. The implant can fail for a number of reasons, including infection of the hip replacement, breakage or wearing out of the implant, and damage to the bone surrounding the implant. The most common reason for replacement joint failure is loosening of the implant.

During surgery, prosthetic joints are either press-fit into the bone, or cemented into place. In both cases, the prostheses are fit tightly into the femur and pelvis so that the implant cannot move. When implants loosen, the hip replacement can begin to move small amounts. This usually occurs because the cement breaks, or because the bone dissolves from around the metal implant or from around the bone cement. Often, implants loosen as a result of a condition known as osteolysis that occurs when fragments created by wear-and-tear on the prosthetic are shed into the area around the joint, causing an inflammatory reaction that induces bone reabsorption. Implant loosening is almost always associated with increased pain and loss of motion for the patient.

For decades, the only treatment for hip replacement loosening is a second hip replacement, known as hip replacement revision surgery. Physicians and patients alike are very concerned about hip replacement loosening because a hip replacement revision surgery is even more difficult, and typically less successful, than the original hip replacement surgery. This is expensive and high risk for elderly patients, many of whom have compromised immune systems, poor muscle tone, cognitive impairment, and osteoporosis. Following revision surgery, patients often recover less overall motion of the joint, and the longevity of replacement joint decreases with each successive replacement. For this reason, physicians tend to avoid joint replacement surgery until absolutely necessary, hoping that the implant will stabilize and can be treated with pain and anti-inflammatory medications. This delay causes chronic discomfort, pain, and reduced stability and range of motion in the patient while he or she waits out the useful lifetime of the failing joint.

It is astonishing that these individuals have no better recourse than to have the implant re-implanted, at great cost, pain, and risk in a high-risk population. This long standing but un-met need emphasizes the lack of an acceptable alternative treatment. Alternative methods for reducing or correcting joint replacement loosening are urgently needed.

Therefore, it is an object of the invention to provide minimally invasive methods of preventing, decreasing, or correcting loosening of existing replacement joints.

### SUMMARY OF THE INVENTION

A non- or minimally invasive method of using a bone cement composition such as a polymethylmethacrylate (PMMA) bone cement or osteobiologic bone cement such as calcium phosphate, calcium sulfate, bioglass or combinations thereof, to secure a hip or other prosthetic implant that has loosened following the initial implant surgery has been developed. The method prolongs the life of the implant, and delays or avoids the need for revision surgery. The method includes delivery of a composition including one or more bone cement components or the formulation alone or in combination with one or more additives and/or fillers to the site of a loosened or loosening joint. Typically, the site is a region between the implant and the adjacent bone or between the bone cement and the adjacent bone. Preferred bone cement can be injected as a liquid or suspension where it then solidifies in situ. Additives include, but are not limited to, biological or bioactive agents, therapeutic agents, diagnostic agents such as X-ray or other contrast media, dyestuffs, stabilizers, and catalyst. In some embodiments, the bioactive components are useful for promoting bone tissue growth around the restorative cement and preferably, bone tissue ingrowth into the cement. The bioactive component can serve as a stiffening and strengthening agent for the bone cement.

The method can be used to treat loosened joints after total arthroplasty, hemi or partial arthroplasty, revisionary surgery, and resurfacing procedures. In the most preferred embodiments, the method is used to treat a loosened hip, knee, shoulder, wrist, finger, ankle, or elbow implant. Typically, the composition is delivered to the site of the loosening implant by injection through a delivery device such a needle, cannula, bone access port, catheter, or, microcatheter. Access may be created or enhanced by drilling into the bone or space between the bone and the implant. Devices have been designed for high pressure delivery of the bone cement into the space between the bone and implant. Percutaneous procedures are preferably carried out using the aid of computer tomography (CT), x-ray guidance, or fluoroscopic visualization. These visual guidance techniques facilitate placement of the delivery device at the proper position within the gap between the bone and the prosthesis, as well as the actual placement of the bone cement composition. In some embodiments, the method of percutaneous joint repair includes a modified kyphoplasty procedure.

### BRIEF DESCRIPTION OF THE DRAWINGS

FIG. 1 is a prospective view of a threaded handled cannula to introduce bone cement into space between bone and prosthetic implant.

FIG. 2 is a prospective view of a prosthetic implant in bone, showing space where implant has loosened, and jig secured to bone to introduce bone cement into space.

FIG. 3 is a prospective view of a high pressure cartridge for administration of bone cement in high pressure gun.

FIG. 4 is a prospective view of a high pressure gun for administration of bone cement into space between bone and loosened prosthetic implant.

### DETAILED DESCRIPTION OF THE INVENTION

### I. Methods of Treating Loosened Joint Implants

A. Compositions
1. Bone Cement

Bone cement is a material used during joint replacement surgery to fix the artificial joints to the skeleton. It acts as a filler, however, not a glue, filling the space between the prosthetic and the bone. Bone cement helps to absorb the forces acting on the implant to ensure that it remains in place over the long term. The human hip, for example, is exposed to forces approximately 10-12 times the body weight of the patient.

Any suitable bone cement known in the art can be used in the disclosed method. In a preferred embodiment, the bone cement is a biodegradable polymer/osteoconductive or osteoinductive filler, such as hydroxyapatite or calcium phosphate. Calcium phosphate cements are materials that typically include calcium phosphate compounds that are capable of self-setting to a hard mass. Exemplary calcium phosphate compounds include, but are not limited to, monocalcium phosphate monohydrate, anhydrous monocalcium phosphate, anhydrous dicalcium phosphate, dicalcium phosphate dehydrate, octacalcium phosphate, α-tricalcium phosphate, β-tricalcium phosphate, amorphous calcium phosphate, hydroxyapatite, tetracalcium phosphate.

Calcium phosphate bone cements typically begin as two components, a powder and a fluid. The powder is usually a mix of monocalcium phosphate monohydrate, tricalcium phosphate, and calcium carbonate, while the fluid is a sodium phosphate solution. Before administration to the patient, the two components are mixed into an injectable paste. After injection, the paste hardens within a few minutes to form a carbonated calcium-phosphate apatite very similar to the mineral phase of bone. The cement interdigitates with adjacent bone as it is injected, forming a solid structure. The properties of various calcium phosphate cements are known in the art. See for example, Chow, Dent Mater. J., 28(1):1-10 (2009); Ambard, et al., Prostodont, 15: 321-328 (2006); Bohner, et al., Biomaterials, 26: 6423-6429 (2005); Lewis, Biomed Mater Res Part B: Appl Biomater, 76B: 456-468 (2006); Chow, Cem Res Prog, Struble, L J (Ed.), The American Ceramic Society, Westerville, Ohio; pp. 215-238 (1998). Exemplary calcium phosphate bone cements include, but are not limited to, NORIAN SRS® (6, 11, 12, 17, 19, 21, 23, 25, 26, 28, 31, 32, 33, 34, 40, 42, 44, 46, 47, 49, 50) (Synthes Inc.), BONESOURCE™ (Stryker Inc.), ALPHA-BSM® (ETEX Corp.), CARRIGEN® (ETEX Corp.), EQUIVABONE® (ETEX Corp.), BETA-BSM® (ETEX Corp.), and GAMMA-BSM® (ETEX Corp.). Other exemplary calcium containing materials include biodegradable porous mixtures of hydroxyapatite and tricalciumphosphate, such as TRICOS® from Biomatlante (France) or CAMCERAM® from Cam Implants, Leiden (Netherlands). Nonporous hydroxyapatite/tricalcium phosphate granules, pure hydroxyapatite granules (porous or nonporous), tricalcium phosphate granules (porous or nonporous), calcium sulfate granules, bone chips (either autograft or allograft) or xenograft bone chips may also be used. KRYPTONITE™ (Bone Cemen, Drikot Medical) is non-toxic, with bone-like mechanical properties composed of naturally occurring fatty acids and calcium carbonate.

In the most preferred embodiments, the bone cement is an injectable bone cement. Injectable calcium phosphate bone cements are described in U.S. Pat. No. 7,892,346 to Insley, et al. U.S. Pat. No. 7,494,950 to Armitage, et al., describes a process for making an implantable composition that is at least partially biodegradable, at least partially resorbable, at least partially biocompatible, or a combination thereof. The process includes incorporating a flow additive with a calcium salt-containing component to form a flowable calcium salt-containing composition that can be injected through a syringe needle. U.S. Publication No. 2010/0305714 by Lee, et al., describes a synthetic bone substitute which may be inserted into bony voids or cavities, and method for preparing such synthetic bone substitute and to a method for filling a cavity in a substrate. Lui, et al., J. Biomedical Mat. Res. Part B. Applied Biomaterials, 94B(1):72-79 (2010) describes an injectable bone cement based on mineralized collagen.

In another embodiment the bone cement is a polymethylmethacrylate (PMMA) bone cement. These are not as preferred since they are brittle and not as osteoinductive. Like calcium phosphate cements, PMMA bone cements typically begin as two components, a powder (i.e., pre-polymerized PMMA and/or PMMA or MMA co-polymer beads and/or amorphous powder, radio-opacifer, initiator) and a liquid (MMA monomer, stabilizer, inhibitor). For example, in some embodiments utilizing a PMMA bone cement, the powder includes a fine bead polymer of methyl methacrylate with a copolymer content of methyl acrylate including 0.5% of dibenzoyl peroxide which acts as an initiator. One or more additives can be about 30% or less by weight, of the solid component. The liquid monomer component of PMMA, methyl methacrylate, typically includes a polymerization accelerator, such as dimethyl-p-toluidine, and a stabilizer, such as hydroquinone. The liquid component can also include dyestuffs and other expedient additives. The two components are mixed and a free radical polymerization of the monomer occurs when the initiator is mixed with the accelerator. As described below, the viscosity of PMMA bone cement can be controlled, for example, for delivery by injection.

In another embodiment, the bone cement is a bioglass. Bioglass bone cements are injectable cements composed of combinations of bioactive glass ceramic powders with resins. Several formulations of cement in which bioactive glass powders are combined with a bisphenol-a-glycidyl methacrylate (Bis-GMA)-based resin have been developed. Other formulations of bioactive cements composed of Bis-GMA-based resins mixed with combeite glass ceramic fillers (e.g. Cortoss®, Orthovita Inc.) have been developed and used for fractures in the extremities and the spine.
2. Bone Cement Additives and Additional Therapeutic Agents

In some embodiments, the bone cement compositions include one or more additives. Additives include, but are not limited to, therapeutic, prophylactic or diagnostic agents such as biological or bioactive agents such as bone morphogenic protein or fibroblast growth factor, X-ray contrast media, such as, for example, barium sulphate or zirconium dioxide, dyestuffs for identification, such as, chlorophyll, N,N-dimethyl-p-toluidine (DMPT) to initiate cold curing, stabilizer, such as hydroquinone, initiator such as benzoyl peroxide, and fillers.

The additive and/or filler can be mixed with the bone cement, or one of the intermediate components, such as the powder or liquid component of PMMA. The additive and/or filler can be inert, or the filler can include a bioactive component. In some embodiments, bioactive components are useful for promoting bone tissue growth around the restorative cement and, preferably, bone tissue ingrowth into the cement. In addition, the bioactive component can serve as a stiffening and strengthening agent for the bone cement. Representative documents describing such materials include U.S. Pat. Nos. 2,920,971, 3,732,087, 3,981,736, 4,652,534, 4,643,982, 4,775,646, 5,236,458, 5,336,642, 5,681,872, and 5,914,356, as well as Brown, W. F., “Solubilities of Phosphate & Other Sparingly Soluble Compounds,” in Environmental Phosphorous Handbook, Ch. 10 (1973).

In some embodiments, the bioactive component is a bioactive glass ceramic, such as CORTOSS™ from Ortho Vita or Bioglass™ (sold by NovaBone), Cervital™, a water-soluble glass, collagen, grafted bone material such as allografts, autografts, and xenografts, calcium phosphate ceramics, or any other bioactive material known to promote bone tissue formation. The bioactive component can include known bioactive materials such as densified and microporous hydroxyapatite, fluorapatite, oxyapatite, wollastonite, apatite/wollastonite glass ceramics, anorthite, calcium fluoride, calcium sulfate, agrellite, devitrite, canasite, phlogopite, monetite, brushite, octocalcium phosphate, whitlockite, cordierite, berlinite, combeite, tetracalcium phosphate, tricalcium phosphate (TCP) (e.g., alpha- and beta-tricalcium phosphates), amorphous calcium phosphate, dicalcium phosphate, phosphoric acid crystals, disodium hydrogen phosphate, and other phosphate salt-based bioceramics. Therefore, in some embodiments the additive and/or filler is a second bone cement.

In some embodiments, the bioactive component is surface modified with one or more coupling groups. Suitable coupling groups include, for example, alkoxysilanes containing epoxide, amine, or vinyl groups, organic isocyanates, acrylic acids, methacrylic acids, polyacrylic acids, citric acids, zirconates, titanates, diamines, amino acids, and polypeptides.

Additives and/or fillers can also include biological, pharmaceutical and/or therapeutic agents, for example, agents in an effective amount to enhance or accelerate bone formation, or enhance bone fusion or bone ingrowth at the site of treatment. Exemplary biological additives used as components of bone cement include bone morphogenic factors (e.g., bone morphogenic proteins (BMP's)), growth factors, bone marrow aspirate, gene delivery vectors (promoting osteogenesis or preventing osteolysis), pluripotent or multipotent cells (which can engineered by gene delivery vectors to upregulate expression of desired proteins such as BMP's), plasmid DNA or RNA or proteins (e.g., bone morphogenetic proteins 2, 4, 7). Examples of biological fillers include, but are not limited to, recombinant BMP-2 (rhBMP-2) (e.g. INFUSE® Bone Graft by Medtronic®, Memphis, Tenn.), LIM mineralization protein-1 (LMP-1), demineralized bone matrix (DBM), growth differentiation factors (GDF), transforming growth factors (TGF), hydroxyapatite, tri-calcium phosphate (TCP), FORTEO® (Eli Lilly and Company) (teriparatide (rDNA origin) injection, which contains recombinant human parathyroid hormone (1-34), agents that bind to rhPTH (1-34), collagen, and alginate.

BMPs are now produced using recombinant DNA technology. Oral and orthopaedic surgery have benefited greatly from commercially available BMP formulations. In regenerative medicine, BMPs are delivered to the site of the fracture by being incorporated into a bone implant, and released gradually to allow bone formation, as the growth stimulation by BMPs must be localized and sustained for some weeks. Currently, two BMPs products have been approved by the Food and Drug Administration (FDA) for clinical applications (fractures of long bones, intervertebral disk regeneration), by delivery in a purified collagen matrix (which is implanted in the site of the fracture). These are Infuse BMP-2 (Medtronic) and OP-1 BMP-7 (Stryker Biotech).

Therapeutic additives can be agents that prevent bone loss such as bisphosphonates (e.g., alendronate), antibiotics such as gentamicin, tobramycin, vancomycin, erythromycin, cefazolin, and clindamycin, anti-inflammatories, pain killers, or combinations thereof.

In some embodiment, the bone cement composition can include a plasticizer, e.g., a cohesion promoter. The presence of the plasticizer may assist the particles to coalesce together to enable the coalesced particles to flow plastically. The plasticizer alters the surface chemistry of the particles and does not affect the setting characteristics of the bone substitute composition to form a cement. In addition, the presence of plasticizer can result in the bone substitute composition having a viscosity which renders it injectable into a site of a patient from about one to five minutes after initiating the mixing procedure of the powder and liquid components. After injection, the injectable composition sets and forms a cement in vivo, e.g., at site of joint repair.

The plasticizer can be selected from a variety of materials known in the art such as sodium dextran sulfate, alpha-cyclodextrin, beta-cyclodextrin, gamma-cyclodextrin, alginic acid or salts thereof, polyvinyl pyrrolidone, hyaluronic acid or salts thereof, chondroitin sulfate, chitosan lactate, hydroxypropyl methylcellulose, carboxymethyl cellulose and mixtures thereof. The plasticizer can be present in the powder component or in the liquid component or both. In a preferred embodiment, the plasticizer is present in the powder component. The plasticizer can be present in various amounts. The amount of plasticizer employed may depend on the specific starting compounds selected for use in the bone cement composition. For example, the plasticizer can be present in an amount of from about 2% to about 10% by weight based on total weight of the powder component without the weight of other filler or additives.

The content of all additives can be optimized within a relatively wide range and depends on the particular profile of requirements of the bone cement or of the corresponding secondary products. For example, the total amount of filler and/or additive may represent from about 10 to about 95% by weight of total cement mix. Preferably an inert filler represents from about 65% to about 85% by weight of total cement mix.

In some embodiments additive and/or filler such as biological and therapeutic agents, are delivered to the site of treatment separately from the bone cement, for example, before or after delivery of the bone cement.

B. Methods of Treatment and Devices for Use Therein
1. Patients to be Treated

The method for treating loosened replacement joints is useful for treating replacement joints that have loosened after arthroplasty. The method can be used to treat loosened joints after total arthroplasty, hemi or partial arthroplasty, revisionary surgery, and resurfacing procedures. In the most preferred embodiment, the method is used to treat a loosened hip prosthesis following total or partial hip replacement, or a hip resurfacing procedure. In other embodiments, the method and compositions are used to treat a loosened prosthesis of another joint, such as a knee, shoulder, wrist, finger, ankle, or elbow.

The method disclosed herein can be used to treat a loosened prosthesis any time after the initial joint replacement, for example, one or more days, weeks, months, or years after the initial surgery. In some embodiments the disclosed method is used to treat a loosened prosthesis subsequent to one or more revision surgeries.

It is believed that bone cement administered to the site of the loosened joint serves as a “filler”, sealing the gap between the prosthetic and the bone and stabilizing the implant and preventing or reducing the pain associated with joint loosening. It is also believed that the procedures increase the longevity of existing implants and reduce the need for replacement or revision surgery. The method can be performed once, or repeated as needed to repair the loosened joint.

In the most preferred embodiments, the method is used to treat prosthetics that were designed to be fixed into place using bone cement during the initial surgery. In an alternative embodiment, the method is used to treat loosened “press-fit” or “cementless” implants, i.e., implants fixed into place without the use of cement during the initial surgery. Typically, cemented prosthetic joints have a smooth surface in contact with the bone while cementless prosthetics have a rough, coated, textured, or porous surface to encourage new bone growth into the implant. It is believed that the method is useful for treating both smooth and textured implants at the point of contact between the prosthetic and the bone or between the bone cement surrounding the prosthesis and the adjacent bone. The method is useful for treatment of loosened prosthetic joints constructed of various materials, including, but not limited plastics, such as polyethylene, metals, such as titanium, stainless steel or cobalt chrome, and ceramics, which are the oxide of a metal, for example, aluminum oxide.

Numerous devices have been developed and are being developed for arthroplastic procedures. Prostheses are commercially available. Modular prosthetic devices useful in total or revision hip procedures are described in U.S. Pat. No. 7,641,698 to Gibbs, et al. Systems for the total replacement of the shoulder joint due to disease or trauma, i.e., a total shoulder arthroplasty, generally replicate the natural anatomy of the shoulder, and typically include a humeral component having a stem which fits within the humeral canal, and an articulating head which articulates within the socket of a glenoid component implanted within the glenoid of the scapula. Examples of shoulder arthroplasty devices are described in U.S. Pat. No. 7,854,768 to Wileley, et al., A typical knee prosthesis includes a femoral component, a patella component, a tibial tray or plateau and a tibial bearing insert coupled to the tibial tray. Examples of knee prostheses are described in in U.S. Pat. No. 4,209,861 to Walker; U.S. Pat. No. 4,298,992 to Burstein et al.; U.S. Pat. No. 4,213,209 to Insall et al.; U.S. Pat. No. 4,888,02 to Forte, et al., U.S. Pat. Nos. 5,171,244, 5,171,276 and 5,336,266, to Caspari et. al., U.S. Pat. No. 4,892,547, to Brown, U.S. Pat. No. 4,298,992 to Burstein et al., and U.S. Pat. No. 6,068,658 to Insall et. al., Four second-generation ankle prostheses with promising results are used currently. The cementless designs include the Scandinavian total ankle replacement (STAR), the Buechel-Pappas total ankle arthroplasty (Endotec, South Orange, N.J.), and the TNK ankle (Nara, Japan). The Agility total ankle system (DePuy, Warsaw, Ind.) is a cemented design (Taljanovic, et al., RadioGraphics, 23(5):12951314 (2003)).
2. Procedures and Devices for Use Therein

The compositions including bone cement are used in a surgical procedure to treat loosened replacement or implanted joints in a patient. The procedure includes delivery of a composition including one or more bone cements alone, or in combination with one or more additives and/or fillers to the site of a loosened implant. Typically, the site is a region between the implant and the adjacent bone. In some embodiments, the region is a gap or void. The procedure can be performed as an open surgical procedure, or a percutaneous surgical procedure. In the most preferred embodiments, the procedure is performed percutaneously with the aid of computer tomography (CT) or x-ray guidance, or fluoroscopic visualization. Percutaneous embodiments typically include making one or more skin incisions over each targeted site, to provide a gateway for delivery of the bone cement composition. In some embodiments, the region to be treated is accessed directly. In some embodiments the region is accessed through the bone, for example when injecting material into the femoral stem. When the region is accessed through the bone, an additional step of puncturing or drilling into the bone at one or more locations may be necessary to create a pathway for percutaneous delivery of the composition. This involves opening a space within the bone by drilling or broaching with or without removing material from inside the bone. This facilitates improved fixation of the prosthesis by injection of a cement into this space within the bone around the loosened prosthesis. Thereafter, a bone cement device is used to introduce the cement into the site of the loosened joint.
a. Preparation of Bone Cement

As described above, a number of different bone cement compositions can be used in the method of treatment disclosed herein. Some bone cements are provided as single fluid component. Many bone cements, such as PMMA and calcium phosphate cements, are provided commercially as two components, typically a powder and a liquid, which are combined by the user prior to in vivo delivery. The two components are mixed by the user immediately prior to use. Different apparatus and methods are available in the art for preparing different bone cements. When the device does not contain a delivery component, the bone cement is transferred into a delivery device such as a syringe for injection into the site of treatment as discussed below. In some embodiments, the bone cement mixing device has an injection device incorporated therein. UK Patent No. GB 2 276 560, WO 95/22402, U.S. Pat. No. 5,876,116, U.S. Published Application No. 2007/0297271, and U.S. Pat. No. 7,320,540 describe devices for mixing and delivering bone cement to a patient.

The viscosity of the bone cement composition changes over time from a runny liquid into a dough-like state and eventually sets into a solid during polymerization or solvent removal. The viscosity and set-time can be tailored for each procedure by the user based on a number of factors including the location of the joint, the nature of the loosening, the construction of prosthetic being repaired, the amount or condition of existing bone cement, and the size of the gap or cavity being filled. Methods for manipulating the viscosity and/or set-time of various bone cements are known in art. For example, factors known to effect the viscosity and the set-time of the bone cement, including but not limited to, the ratio of powder/liquid components, the amount of fillers, such as accelerator and stabilizer, method of mixing, and atmospheric conditions such as oxygen concentration, humidity, and ambient and body temperatures (Boger, et al., Eur Spine J., 18(9): 1272-1278 (2009). The preferred viscosity of the cements used for this technique is low enough to allow the cement to be pumped into the space created by the loosening within the bone.

The viscosity and set-time of the bone cement also affects cement leakage, a possible complication associated with the method. The user should apply the bone cement composition to the joint with a viscosity that minimizes bone cement leakage out of the joint. Bone cement leakage can result in irritation or damage, such as thermal damage caused by exothermic solidification of the bone cement, to the tissue surrounding the joint. Leakage can result in pain or abnormal sensation in the patient. The user can reduce the chance of leakage by monitoring the viscosity of the bone cement prior to and during the procedure and by X-ray (fluoroscopic, CT, (etc.) monitoring to detect any leakage before it reaches significant levels. Methods for measuring cement viscosity are known in the art and include visual assessment of the flow characteristics of freshly exposed cement from the tip of a syringe by the user, and rheometer systems such as those described in Boger, et al., Eur Spine J., 18(9): 1272-1278 (2009).
b. Anesthetic

Patients are typically given an anesthetic or analgesic prior to beginning the procedure. The anesthetic can be local, epidural, or general anesthetic. In the most preferred embodiments, patients undergoing a percutaneous procedure are typically given local anesthetic alone or in combination with light sedation. The specific type or combination of anesthetics alone or in combination with sedatives will depend on factors including, but not limited to, the location and duration of procedure, whether the procedure is performed openly or percutaneously, and any pre-existing conditions of the patient, and can be determined using techniques known in the art.

Anesthetic can also be injected with the bone cement. In these embodiments, a solution containing a local anesthetic is used to wet the bone cement components. In another embodiment, the bone cement components are mixed without an anesthetic, and the anesthetic is then added to the prepared bone cement. Alternatively, the anesthetic can be injected separately, either before or after the injection of the bone cement. Preferably, the anesthetic is injected prior to, or simultaneously with, the injection of the bone cement.
c. Delivery

During the procedure, bone cement is placed into the region between the loosened prosthetic and the patient's bone which may be in a space between the metal stem and bone or between the bone cement and bone. In the most preferred embodiment, the composition including cement is injected into the region. For example, if the procedure is being used to correct a loosened hip implant, bone cement can be injected between the acetabulum and the acetabular cup of the prosthesis and/or between the fermoral stem and the interior of the femur where the fermoral stem resides. Some implants are fixed into place during the initial replacement procedure using bone cement. Over time, the bone cement used in the initial procedure can crack or break, contributing to the loosening of the joint. In some embodiments, the procedure described herein includes visualizing the defects and voids around the loosened implant and then injecting bone cement to fill or seal cracks and gaps in the existing or residual bone cement from to the initial or revisionary implant procedures. In some embodiments, lavage is performed preferably under suction, to remove debris such as fragments of bone, fragments of deteriorated bone cement, and/or fragments of implant from the region and/or gap to be filled.

Typically, the composition is delivered to the site of the loosened implant through a delivery device such a needle, cannula, bone access port, catheter, or, microcatheter. The composition is typically injected through the needle, cannula, bone access port, or catheter, using suitable injection system. Suitable injection systems are known in the art and include, but are not limited to, simple syringes connected directly or indirectly to the needle or cannula, which can be used to inject the cement by applying manual force, or injection devices that utilize force amplification allowing higher forces being applied to the cement during injection.

Selection of the delivery device is determined based on a variety of factors including but not limited to, the location of the joint, the size of the gap to be filled, the amount of the composition to be delivered, and the maximum injectable viscosity of the composition, and the delivery and injection devices being used. It is believed that the maximum injectable viscosity is inversely proportional to syringe diameter (Boger, et al., Eur Spine J., 18(9): 1272-1278 (2009)). For example, the equivalent force required to inject cement at a particular viscosity relates quite well to the proposed limit of 150 N for syringe-type injections of PMMA (Bohner, et al., Biomaterials, 24(16):2721-30 (2003)). The viscosity of the composition is preferably a flowable solution that can be delivered through an appropriately sized catheter or syringe needle. For delivery through a microcatheter, a viscosity in the range of about 10 to 50 cp is desirable. The viscosity can be substantially higher for delivery through a syringe needle, such as, for example 20 to 300 cp without mechanical assistance or 100 to 500 cp with mechanical assistance. The viscosity will generally be controlled by the molecular weight of the macromers, the solids content of the solution, and the type and amount of fillers (if any). The solids content of the solution will preferably range from about 2 percent by weight to about 30 percent by weight, desirably from about 6 to 12 percent by weight. In the preferred embodiment, the composition should be injected before substantial crosslinking or hardening of the composition has occurred. This prevents blockage of the syringe needle or catheter with gelled/hardened composition.

Suitable devices for use in injecting the bone cement are shown in the attached Figures. FIG. 1 is a simple device 10 for drilling a hole into the bone to allow insertion of cement. The device 10 include a hollow core 12, threads 14 to facilitate drilling into the bone, and a handle 16.

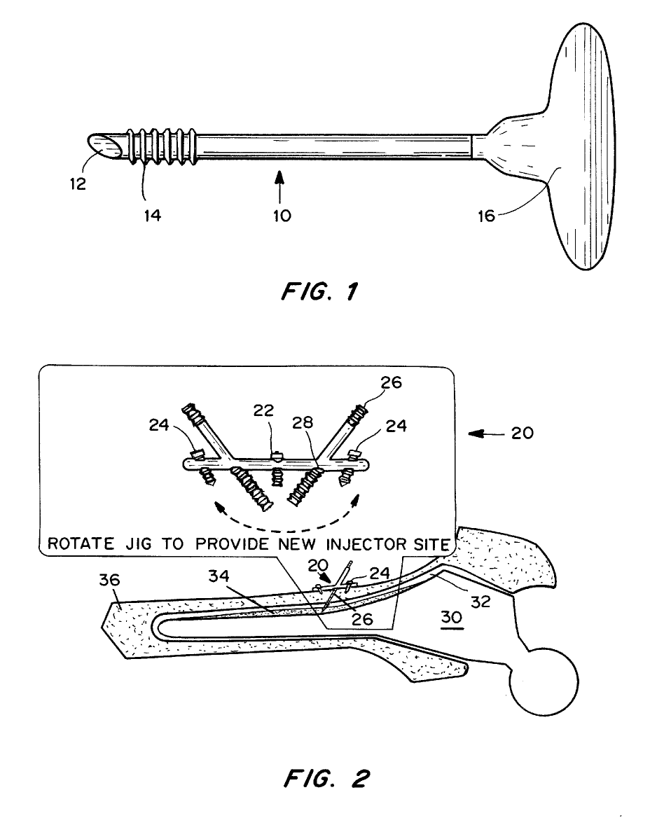

FIG. 2 is a diagram showing a more complex jig 20, secured to the bone cortex 36 using set screw 22, which can be rotated at the site to the desired injection site, and angled set screws 24, then the cement injector 26 inserted through jig 28 into the space 34 between the implant 30 and the bone cortex 36 so that cement 32 can be filled into the space 34.

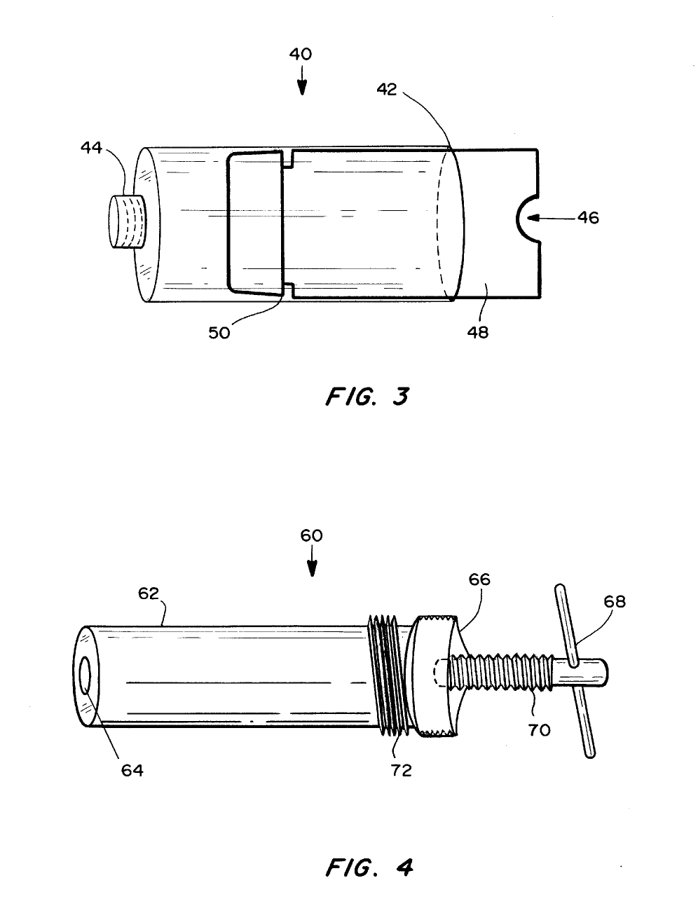

FIG. 3 shows a high pressure cartridge 40 for use in a high pressure cement gun, since in some embodiments the bone cement will be viscous and therefore difficult to place within the space next to the implant. The cartridge 40 includes a syringe housing 42, luer lock top 44 for connection to a needle or cannula, dimple 46 for high pressure plunger, extra strong solid plunger 48, and plunger tip 50.

As shown in FIG. 4, the cartridge 40 is inserted into a high pressure cement gun 60 including a metal cylinder 62 to accept the cartridge 40, having an opening 64 for the luer lock tip 44, onto which the threaded cap/pressurizer 66 is threaded via threads 72. The plunger 48 is then depressed by turning the handle 68 so that the threads 70 engage the cap 66 and applies pressure to the plunger 48 at the dimple 46.

These devices can be provided separately in sterile form for use by the physician, in a kit including the bone cement composition components in a separate vial (or vials) or pre-loaded for use by the physician.

Once the bone cement delivery device is placed at the desired location within the bone, the bone cement can be injected. After the desired amount of bone cement has been injected, the bone cement delivery device can be removed from the bone site as the bone cement is allowed to cure. The incision in the patient can be repaired by stitching and/or bandaging. The user can confirm the gap is filled and the joint repaired by x-ray projection, fluoroscopic views, or by other methods known in the art.
d. Visualization

Percutaneous procedures are preferably carried out using the aid of computer tomography (CT) or x-ray guidance, or fluoroscopic visualization. These visual guidance techniques facilitate placement of the delivery device at the proper position within the gap between the joint and the prosthesis, as well as the actual placement of the delivered bone cement composition. In some embodiments, the cement contains radiopaque materials or other contrast agents so that when injected, cement localization and leakage can be observed. Suitable contrast agents to be used in combination with bone cement are known in the art. The visualization of cement injection and extravasion are important in allowing the user to halt or terminate injection when leakage is evident. The introduction of cement material can be imaged several times, or continuously, during the treatment depending on the imaging method.
e. Optional Procedures

In some embodiments, the method of percutaneous joint repair optionally includes a modified kyphoplasty-like procedure. Kyphoplasty is a variation of a vertebroplasty that attempts to stop the pain caused by the bone fracture and attempts to restore the height and angle of kyphosis of a fractured vertebra (of certain types), followed by its stabilization using injected bone filler material. In the modified kyphoplasty-like procedure used in the method for prosthetic joint repair, typically, a small balloon is deployed between the prosthetic and the existing bone to create or increase the void or gap between implant and bone prior to cement delivery. Once the void is created, the procedure continues as described above, but includes delivering bone cement to the newly created void. Methods of balloon kyphoplasty are known in the art, see for example, Balloon Kyphoplasty, Becker and Ogon (Eds.), Springer-Verlag/Wien, New York (2008).

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

We claim:

1. A minimally invasive method of stabilizing a prosthetic implant in a bone comprising delivering an effective amount of a bone cement composition to a space between a prosthetic bone implant and the adjacent bone formed by loosening of the implant, bone loss or degradation of bone cement placed at the time of implantation, to secure the loosened implant to the adjacent bone.

2. The method of claim 1 wherein the bone cement is selected from the group consisting of calcium phosphate bone cement, polymethylmetacrylate (PMMA) bone cement, and bioglass bone cement.

3. The method of claim 1 wherein the bone cement composition is a liquid.

4. The method of claim 1 wherein the bone cement composition further comprises a bioactive, therapeutic, prophylactic or diagnostic additive.

5. The method of claim 1 wherein the composition is delivered to the region percutaneously.

6. The method of claim 5 comprising delivering the bone cement composition through a delivery device selected from the group consisting of a needle, cannula, bone access port, catheter, and microcatheter.

7. The method of claim 1 further comprising lavaging the space between the bone and prosthetic implant prior to delivering the bone cement composition into the space.

8. The method of claim 1 further comprising performing a modified kyphoplasty-like procedure.

9. The method of claim 1 wherein the joint is selected from the group consisting of hip, knee, shoulder, wrist, finger, ankle, and elbow.

10. The method of claim 9 wherein the joint is a hip.

11. A device for minimally invasive stabilization of a prosthetic implant in a bone by delivering an effective amount of a bone cement composition to a space between a prosthetic bone implant and the adjacent bone formed by loosening of the implant, bone loss or degradation of bone cement placed at the time of implantation, to secure the loosened implant to the adjacent bone, the device comprising
A cannula with a threaded tip and a handle, in a kit comprising the bone cement formulation.

12. A device for minimally invasive stabilization of a prosthetic implant in a bone by delivering an effective amount of a bone cement composition to a space between a prosthetic bone implant and the adjacent bone formed by loosening of the implant, bone loss or degradation of bone cement placed at the time of implantation, to secure the loosened implant to the adjacent bone, the device comprising
A jig including stabilization means at each end and a housing for a cement injector positioned at an angle extending outwardly from the jig,
Wherein the cement injector can be inserted into and through the housing, and
The jig can be rotated about the stabilization means at a first end of the jig, and secured at the second end of the jig.

13. The device of claim 12 in a kit including a bone cement composition.

14. A cement gun for use in minimally invasive stabilization of a prosthetic implant in a bone by delivering an effective amount of a bone cement composition to a space between a prosthetic bone implant and the adjacent bone formed by loosening of the implant, bone loss or degradation of bone cement placed at the time of implantation, to secure the loosened implant to the adjacent bone,
The gun comprising a housing for a cartridge having a tip for extruding bone cement into the site in need thereof,
the housing having at one end an opening for the cartridge tip,
the housing having a securable cap at the other end,
wherein the cap has a rotatable handle applying pressure to the cartridge containing the bone cement composition to extrude bone cement from the cartridge through the tip.

15. The cement gun of claim 14 further comprising a cartridge for placement in the gun, the cartridge comprising a plunger and plunger tip, the plunger comprising a dimple for acceptance of the handle as it applies pressure to the cartridge.

16. The cement gun of claim 15 wherein the cartridge comprises a bone cement formulation or is in a kit comprising the bone cement formulation.

17. A sterile disposable kit for use in minimally invasive stabilization of a prosthetic implant in a bone by delivering an effective amount of a bone cement composition to a space between a prosthetic bone implant and the adjacent bone formed by loosening of the implant, bone loss or degradation of bone cement placed at the time of implantation, to secure the loosened implant to the adjacent bone, comprising the bone cement in a dosage unit suitable for filling a defined space.
